(19) United States
(12) Patent Application Publication
(10) Pub. No.: US 2025/0002438 A1
(43) Pub. Date: Jan. 2, 2025

(54) POTASSIUM CATALYSTS FOR DEHYDRATION OF LACTIC FEEDS

(71) Applicant: LAKRIL TECHNOLOGIES CORP., Chicago, IL (US)

(72) Inventors: Christopher P. Nicholas, Evanston, IL (US); Benjamin R. Hoekstra, Chicago, IL (US); Zachary T. Pursell, Homewood, IL (US); Lyle E. Monson, Schaumburg, IL (US); Collette L. Nicholas, Evanston, IL (US)

(21) Appl. No.: 18/760,666

(22) Filed: Jul. 1, 2024

Related U.S. Application Data
(60) Provisional application No. 63/524,273, filed on Jun. 30, 2023.

(51) Int. Cl. C07C 51/377; B01J 29/08; B01J 29/60; B01J 35/54; B01J 37/00; B01J 37/08; B01J 37/30

(52) U.S. Cl. CPC C07C 51/377; B01J 29/08; B01J 29/60; B01J 35/54; B01J 37/0018; B01J 37/08; B01J 37/30; B01J 2229/186

(57) ABSTRACT

The present disclosure sets forth a proposed solution to decarbonize the acrylic chemicals industry with a lactic-to-acrylic technology producing bio-based acrylics that are sustainable and eco-friendly and are at cost parity with petrochemicals. In the present disclosure, catalysts comprising potassium and zeolites were relied on as the catalyst base. It has been found high yield lactic-to-acrylic technology of the present disclosure is industrially feasible at new or as an add-on to existing bio-refining facilities throughout the Midwest due to the high yields achieved.

### RELATED APPLICATIONS

This patent application claims priority to and benefit of U.S. Provisional Application No. 63/524,273, filed Jun. 30, 2023, which is herein incorporated by reference in its entirety.

### REFERENCE TO GOVERNMENT RIGHTS

This invention was made with government support under DE-SC0022612 awarded by The U.S. Department of Energy and The Advanced Manufacturing Office of the Office of Energy Efficiency and Renewable Energy. The government has certain rights in the invention.

### FIELD

The field is the catalytic dehydration of lactic acid to acrylic acid.

### BACKGROUND

Acrylic acid and its alkyl acrylate derivatives are high volume chemicals at the base of the paints, coatings, adhesives, and superabsorbent polymer industries. Acrylics are currently produced from oxidation of petroleum-derived propylene by two step oxidation via acrolein over bismuth molybdate catalysts. Decarbonizing industrial sectors has been recognized by many as a critical step toward achieving a livable climate future. Among the many industrial sectors, the National Academies of Science recently identified a low-cost transition to a lower carbon chemical base by 2030 as a key need. A bio-based, sustainable route to these high-volume chemicals would be desirable, provided cost for such a process is economically competitive. One path to bio-based, sustainable acrylic acid is the dehydration of lactic acid or lactate compounds.

The dehydration of lactic acid has been studied in the gas phase over the last 15 years directed towards finding catalyst formulations and/or process innovations to achieve commercialization routes to bio-acrylics without success. Both dehydration catalysts and integrated processes for lactic-to-acrylic and improved fermentation yields and integrated processes for 3-hydroxypropanoic acid (3HP) to acrylic have been studied, but yields of acrylics are usually less than 75-80% and have low space time yield. An improved catalyst comprises a solid acid and a multifunctional flexible modifier comprising a functional group such as an amine which has shown 92% yield. This yield was obtained at 0.76 volume percent methyl lactate in the N2 feed, leading to a low space-time yield. Further, amines are difficult to add to inorganic catalyst formulations. An organic solvent such as methanol is often required, adding cost to the catalyst preparation. Additionally, the amine may or may not adhere well to the catalyst during catalysis, potentially requiring recovery downstream and reimpregnation of the amine onto the catalyst to maintain performance.

This disclosure is directed to catalyst formulations that provide the desired selectivity performance of an amine containing catalyst but without the complications of adding an amine.

### BRIEF SUMMARY

The present disclosure sets forth a proposed solution to decarbonize the acrylic chemicals industry with a lactic-to-acrylic technology producing bio-based acrylics that are sustainable and eco-friendly and are at cost parity with petrochemicals. Corn provides a viable plant-based sugar source for production of bio-based acrylic as a high-volume chemical sustainably and economically through fermentation pathways to lactic acid. The high yield lactic-to-acrylic technology of the present disclosure harnesses the output of these existing regionally installed biorefineries to produce a sustainable, low carbon acrylic acid. In the present disclosure, K⁺ exchanged FAU zeolites were relied on as the catalyst base to understand the impact of cation on the conversion, selectivity, and longevity of the catalyst while maintaining high yields of acrylic product. Additional methods of exchanging K⁺ into the FAU zeolite are further disclosed.

A catalyst formulation for dehydration of lactic sources comprises a zeolite and at least one of one or more metal oxide sources, one or more potassium ion sources, and one or more sodium ion sources. The zeolite and at least one of one or more metal oxide sources, one or more potassium ion sources, and one or more sodium ion sources may be combined to form a solid acid catalyst. The solid acid catalyst may comprise surfaces defining pores and a multiplicity of acid sites on the surfaces. A total cation to aluminum ratio may be greater than 1.0 and less than 1.5.

The catalyst formulation may be absent an amine. The zeolite may comprise pore openings delimited by 12-membered rings. The catalyst formulation may have a K/Al ratio greater than about 0.01 and less than 1.0. In examples, an XRD peak ratio for the catalyst may be greater than 1.00 and less than about 1.65. In examples, an XRD peak ratio for the catalyst may be greater than 1.10. The catalyst formulation may have a Na/Al ratio greater than 0.5 and less than 1.65. The catalyst formulation may have a Si/Al ratio greater than 2.5 and less than 7.0. The zeolite of the catalyst formulation may comprise a FAU zeolite, an LTL zeolite, or a mixture thereof. In examples, the zeolite may be about 25% or more of the catalyst formulation as determined by XRD Crystallinity. The catalyst formulation may be in an extruded form.

A process of producing an acrylic product is also disclosed. The process comprises contracting a lactic source with any of the above examples of the catalyst formulations or combinations thereof. The process further comprises recovering the acrylic product therefrom.

A method for production of a bio-based acrylic acid. The method comprises the step of dehydrating a reactant to yield a product. The step of dehydrating may comprise contacting a feedstream with a catalyst of the above examples of the catalyst formulations or combinations thereof. The feedstream may further comprise a lactic source and water. Specifically, a method for production of a bio-based acrylic acid comprises the step of dehydrating a reactant to yield a product by contacting a feedstream with a catalyst comprising a zeolite and at least one of one or more metal oxide sources, one or more potassium ion sources, and one or more sodium ion sources combined to form a solid acid catalyst comprising surfaces defining pores and a multiplicity of acid sites on the surfaces, wherein a total cation to aluminum ratio is greater than 1.0 and less than 1.5 and the feedstream comprises a lactic source and water. In examples, the feedstream may comprise less than about 50 wt % water. In examples, the feedstream may comprise greater than about 50 wt % water. The product may be one or more of an acrylic acid, an alkyl acrylate, or a cation-balanced acrylate.

The method for production of a bio-based acrylic acid may further comprise the step of obtaining the lactic source from fermentation of sugar sources. The fermentation of sugar sources may produce a mixture of two isomers or a pure lactic acid. The method for production of a bio-based acrylic acid may additionally, or alternatively, further comprise the step of contacting the catalyst with a second feedstream. The second feedstream may comprise an alcohol. The second feedstream may be combined with the first feedstream into a single feedstream for contacting with the catalyst.

In the methods for production of a bio-based acrylic acid the product stream may be formed at a temperature from about 200° C. to about 350° C. by a conversion of the lactic source. The conversion of the lactic source may be greater than about 90 mol %. In examples, the yield of the above-mentioned examples of acrylic acid may be greater than about 84%.

The foregoing and other objects, features, and advantages of the examples will be apparent from the following more detailed descriptions of particular examples as illustrated in the accompanying drawings and tables.

### BRIEF DESCRIPTION OF THE DRAWINGS

FIG. 1 shows an example integration of XRD FAU material. Results from integration of the highlighted peaks are tabulated in Table 2.

FIG. 2 shows the result of catalytic dehydration of methyl lactate to acrylics over commercial Na-FAU zeolites of Example 7. Conversion is shown in open symbols, and dehydration selectivity shown in closed symbols. Na-FAU zeolites from Zeolyst (circles out to 600 min TOS), Tosoh (triangles to 300 min), Zeolyfe (triangles out to 300 min TOS) and Sigma Aldrich 13× molecular sieves (dark box with hash mark is conversion, x markers are selectivity out to 500 min TOS) are shown.

FIG. 3 shows replicate reaction activity measurements during methyl lactate conversion (Part A) over Zeolyst Na-FAU of Example 8 in the Plant A microreactor. Dehydration selectivity is shown in Part B. Data points overlay on top of each other in both plots.

FIG. 4 shows the results of Example 9 methyl lactate conversion in Part A over Na-FAU ion exchanged with K+ to varying extents as described in Example 3. Open circles are the starting Na-FAU, filled circles 1×K+ exchange, open squares 2×K+ exchange, filled squares 3×K+ exchange, open triangles 4×K+ exchange, and box with star the fully exchanged 5×K-FAU material. Part B shows dehydration selectivity where open circles are the starting Na-FAU, filled squares 1×K+ exchange, open circles 2×K+ exchange, filled circles 3×K+ exchange, filled triangles 4×K+ exchange, and filled diamonds the fully exchanged 5×K-FAU material.

FIG. 5 shows XRD scans showing peak ratio differences and loss of FAU (444) peak after K ion-exchange in catalyst formulations prepared by ion-exchange and other catalysts of the application. FIG. 5A shows the end members of the series, the starting Na-FAU and the fully exchanged K-FAU. FIG. 5B shows XRD patterns of Formulation 59, the 1×K ion-exchanged FAU, the fully 5×K ion-exchanged FAU, Formulation 32, and Na-FAU.

FIG. 6 shows additional XRD patterns of catalyst formulations of the application.

FIG. 7 shows catalytic dehydration of methyl lactate to acrylics over K-FAU (dark box with hash mark is conversion, x markers are selectivity) and K-LTL (squares) compared to the performance of base Na-FAU zeolite (circles) from Example 10. Conversion is open symbols, dehydration selectivity closed. Both potassium-containing zeolites achieved superior dehydration selectivity relative to Na-FAU.

FIG. 8 shows catalytic dehydration of methyl lactate to acrylics over several catalysts. Part A shows Formulation 35 (squares) and Formulation 20 (box w hashmark is conversion, x is selectivity) against Na-FAU (circles). Open symbols are conversion, filled show selectivity. Part B shows long lifetime achieved over Formulation 35 (squares, open are conversion, filled selectivity) at the same selectivity as 44TMPD impregnated FAU from Example 5 (open circles are conversion, open triangles selectivity) and K-FAU from Example 4 (box w hashmark is conversion, x is selectivity).

FIG. 9 shows catalytic dehydration of methyl lactate over catalysts discussed in Example 14. Formulation 36 is shown in gray circles with K-FAU zeolite shown with squares with X while Formulation 35 is shown in open circles.

FIG. 10 shows catalytic dehydration of methyl lactate versus ethyl lactate over Formulation 35 (Part A) and Formulation 20 (Part B) as discussed in Example 16. In each part, conversion of methyl lactate is shown in box with hashmark, conversion of ethyl lactate in open circles, methyl lactate dehydration selectivity with x markers, and ethyl lactate dehydration selectivity in closed circles.

FIG. 11 shows catalytic dehydration of methyl lactate versus ethyl lactate over Na-FAU (part A) or K-FAU (Part B) from Example 16. In each part, conversion of methyl lactate is shown in box with hashmark, conversion of ethyl lactate in open circles, methyl lactate dehydration selectivity with x markers, and ethyl lactate dehydration selectivity in closed circles.

FIG. 12 shows catalytic dehydration of methyl lactate versus ethyl lactate over K-LTL from Example 16. Conversion of methyl lactate is shown in box with hashmark, conversion of ethyl lactate in open circles, methyl lactate dehydration selectivity with x markers, and ethyl lactate dehydration selectivity in closed circles.

FIG. 13 shows catalytic dehydration of ethyl 3-hydroxypropanoate (E3HP) over Formulation 35 from Example 18. E3HP was prepared as 5 weight-% and 80 weight-% feedstocks in water. Conversion of 5 wt % solution is shown in open circles and dehydration selectivity in boxes with X. Conversion of 80 wt % solution is shown in filled triangles and dehydration selectivity in filled diamonds.

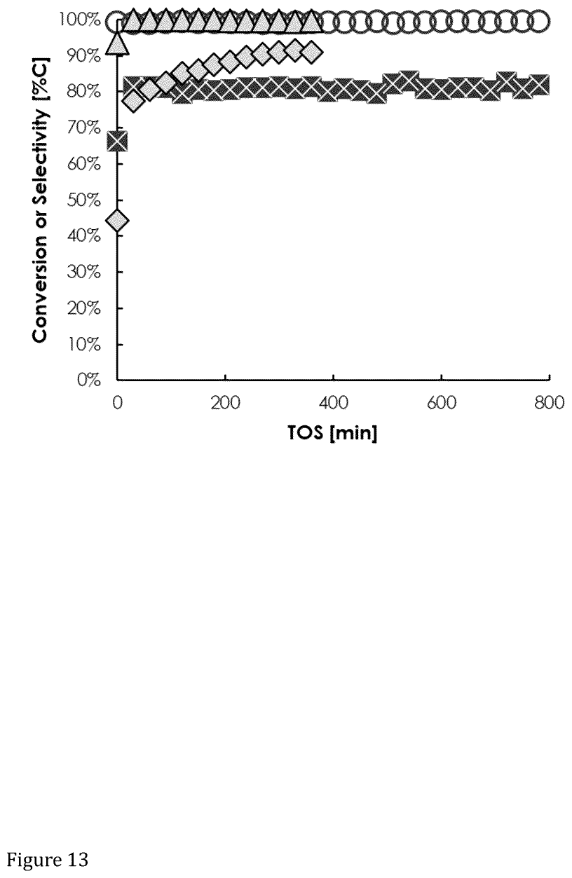

FIG. 14 shows the catalytic dehydration of 5 weight-% methyl lactate in water over Formulation 35. Conversion is shown in filled circles and dehydration selectivity in filled triangles.

FIG. 15 shows catalytic dehydration of methyl lactate to acrylics over formulations with varying ratios of zeolite (Z) to binder. Conversion is shown in circles and dehydration selectivity in triangles. Part A shows conversion of methyl lactate over Formulations 20 (darker circles and triangles) and 21 (lighter circles and triangles). Part B shows conversion of methyl lactate over Formulation 35 (darkest circle and triangle), 56 (medium circle and triangle) and 59 (lightest circle and triangle). Dehydration selectivity was not impacted; however, methyl lactate 100% conversion run time decreased as the zeolite content decreased.

FIG. 16 shows catalytic dehydration of methyl lactate to acrylics over alkali-based formulations with varying ratios of binder components at fixed zeolite content. Conversion is shown in circles and dehydration selectivity in triangles. Part A shows Formulation 35 (darkest circles and triangles), 61 (medium circles and triangles) and 60 (lightest). Part B shows Formulation 63 (darkest circles and triangles with long life), 59 (medium circles and triangles), and 62 (lightest circles and triangles with short life and low selectivity) as K/Al ratio changes.

### DEFINITIONS

As used herein, the term “predominant” or “predominate” means greater than 50%, suitably greater than 75% and preferably greater than 90%.

As used herein, the term “substantially” can mean an amount of at least generally about 70%, preferably about 80%, and optimally about 90%, by weight, of a compound or class of compounds in a stream.

As used herein, the term “stream” can include various hydrocarbon molecules and other substances. Moreover, the term “stream comprising Cₓ hydrocarbons” or “stream comprising Cₓ olefins” or “stream comprising Cₓ oxygenates” can include a stream comprising hydrocarbon or olefin molecules or oxygenates, respectively, with “x” number of carbon atoms, suitably a stream with a majority of hydrocarbons or olefins, respectively, with “x” number of carbon atoms and preferably a stream with at least 75 wt % hydrocarbons or olefin molecules or oxygenates, respectively, with “x” number of carbon atoms. Moreover, the term “stream comprising Cₓ+ hydrocarbons” or “stream comprising Cₓ+ oxygenates” can include a stream comprising a majority of hydrocarbon or oxygenate molecules, respectively, with more than or equal to “x” carbon atoms and suitably less than 10 wt % and preferably less than 1 wt % hydrocarbon or oxygenate molecules, respectively, with x−1 carbon atoms. Lastly, the term “Cₓ-stream” can include a stream comprising a majority of hydrocarbon or oxygenate molecules, respectively, with less than or equal to “x” carbon atoms and suitably less than 10 wt % and preferably less than 1 wt % hydrocarbon or oxygenate molecules, respectively, with x+1 carbon atoms.

As used herein, the term “zone” can refer to an area including one or more equipment items and/or one or more subzones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “vapor” can mean a gas or a dispersion that may include or consist of one or more hydrocarbons.

As used herein, the term “overhead stream” can mean a stream withdrawn at or near a top of a vessel, such as a column.

As used herein, the term “bottom stream” can mean a stream withdrawn at or near a bottom of a vessel, such as a column.

As depicted, process flow lines can be referred to interchangeably as, for example, lines, pipes, feeds, gases, products, discharges, parts, portions, or streams.

As used herein, “bypassing” with respect to a vessel or zone means that a stream does not pass through the zone or vessel bypassed although it may pass through a vessel or zone that is not designated as bypassed.

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottom stream back to the bottom of the column. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column.

As used herein, “taking a stream from” means that some or all of the original stream is taken.

As used herein, “space-time yield” or “STY” means the product yield per unit volume of catalyst and per unit time.

As used herein, “lactic” or “lactic source” indicates lactic acid, a lactate derivative thereof, 3-hydroxypropanoic acid (3-HP), a 3-hydroxypropionate derivative thereof, or combinations thereof. More properly, lactic acid is 2-hydroxypropanoic acid and comprises two enantiomers, the R and S. Ethyl lactate is one example of an alkyl lactate derivative and is the ethyl ester of lactic acid. Potassium lactate is an example of a cation-balanced lactate.

As used herein, “reactant” indicates the lactic source present within a stream, whether a feedstream or a product stream or a recycle stream or other stream available within the process.

As used herein, “acrylic” or “acrylic product” indicates acrylic acid, an acrylate derivative thereof, or combinations thereof. More properly, acrylic acid is propenoic acid and is achiral. Ethyl acrylate is one example of an alkyl acrylate derivative and is the ethyl ester of acrylic acid. Potassium acrylate is an example of a cation-balanced acrylate.

### DETAILED DESCRIPTION

The disclosure provides a process for dehydration of a lactic source comprising one or more of a lactic acid, an alkyl lactate, a cation-balanced lactate, 3-hydroxypropanoic acid, an alkyl 3-hydroxypropionate, a cation-balanced 3-hydroxypropionate or a combination thereof. Lactic acid may undergo acid-catalyzed heterolytic decarbonylation, homolytic decarboxylation, and/or heterolytic dehydration. Dehydration of lactic sources leads to acrylic acid formation. The disclosure identifies potassium containing catalyst formulations that provide comparable, and in some instances, identical selectivity performance to prior amine containing catalyst formulations. The present disclosure further identifies potassium containing catalyst formulations as a suitable alternative to amine containing catalyst formulations and that do not present the complications of adding an amine to a catalyst formulation. Potassium and zeolite ratios and various formulations, including several zeolite types and potassium sources, are supported herein, but are not meant to limit the present disclosure thereto. Acrylic acid and its alkyl acrylate derivatives are desired to form polymers used within the paints, coatings, adhesives, and superabsorbent polymer industries. High space-time yields of bio-based acrylic acid are desired to lower cost of production.

The process for dehydration may comprise contacting a feedstream with a catalyst. The feedstream may comprise a lactic source. Lactic sources may comprise one or more of a lactic acid, an alkyl lactate, a cation-balanced lactate, 3-hydroxypropanoic acid, an alkyl 3-hydroxypropionate, a cation-balanced 3-hydroxypropionate or combinations thereof.

Lactic acid or derivatives thereof may be obtained from fermentation of sugar sources. Lactic acid production methods by fermentation may be preferred over chemical synthesis alternatives that produce a mixture of both R and S isomers. The product of microbial fermentation depends on the organism used. Microbial fermentation can result in a mixture of two isomers or in a stereospecific form of optically pure lactic acid. The desired stereospecificity of the product depends on the intended use. Chirality of the lactic acid may not be important for dehydration.

The lactic source may comprise one or more of a lactic acid, a lactate balanced by a Group I cation or ammonium, a lactate balanced by a Group II cation, an alkyl lactate, or a combination thereof. The Group I cation may comprise one or more of lithium, sodium, potassium, cesium, or combinations thereof. The Group II cation may comprise one or more of magnesium, calcium, strontium, barium, or combinations thereof. The alkyl group may comprise a C₁-C₄ alkyl group such as but not limited to, for example, methyl, ethyl, isopropyl, and butyl. The alkyl lactate may comprise one or more of methyl lactate, ethyl lactate, butyl lactate, or combinations thereof.

The feedstream may comprise water. The feedstream may comprise less than 50 wt % water, or less than 25 wt % water or less than 10 wt % water. The feedstream may comprise greater than 50 wt % water, or greater than 75 wt % water, or greater than 90 wt % water.

Dehydrating the reactant includes contacting the feedstream with a catalyst. The catalyst may be a solid acid catalyst, which may include surfaces defining pores and a multiplicity of acid sites on the surfaces. The acid sites may be Bronsted acid sites or ion-exchanged acid sites. The reactant may be dehydrated to yield a product comprising one or more of acrylic acid, alkyl acrylate or a cation-balanced acrylate, or a combination thereof.

The catalyst may comprise a zeolite. Zeolites are crystalline aluminosilicate compositions which are microporous and which are formed from corner sharing AlO₂ and SiO₂ tetrahedra. Numerous zeolites, both naturally occurring and synthetically prepared, are used in various industrial processes. Synthetic zeolites are prepared via hydrothermal synthesis employing suitable sources of Si, Al and structure directing agents such as alkali metals, alkaline earth metals, amines, or organoammonium cations. The structure directing agents reside in the pores of the zeolite and are believed to be largely responsible for the particular structure that is ultimately formed. These species balance the framework charge associated with aluminum and can also serve as space fillers. Zeolites are characterized by having pore openings of uniform dimensions, having a significant ion exchange capacity, and being capable of reversibly desorbing an adsorbed phase which is dispersed throughout the internal voids of the crystal without significantly displacing any atoms which make up the permanent zeolite crystal structure. Zeolites can be used as catalysts for hydrocarbon conversion reactions, which can take place on outside surfaces as well as on internal surfaces within the pore.

Over 250 different zeolite topologies are recognized today. As used herein, zeolites may be referred to by proper name, such as Zeolite Y or LZ-210, or by structure type code, such as FAU. These three-letter codes indicate atomic connectivity and hence pore size, shape, and connectivity for the various known zeolites. The list of these codes may be found in the Atlas of Zeolite Framework Types, which is maintained by the International Zeolite Association Structure Commission at http://iza-structure.org/databases/. One such topology is FAU, often referred to as faujasite after a natural mineral composition of the topology.

Channel systems for known zeolites are described in the Atlas of Zeolite Framework Types as having zero-dimensional, one-dimensional, two-dimensional or three-dimensional pore systems. A zero-dimensional pore system has no pore system running through the zeolite crystal, instead only possessing internal cages. A one-dimensional pore system contains a pore delimited by 8-membered rings or larger that run substantially down a single axis of a crystal. Two-dimensional pore (channel) containing zeolites contain intersecting pores that extend through two-dimensions of a zeolite crystal, but travel from one side of the third dimension of the zeolite crystal to the other side of the third dimension is not possible, while zeolites containing three-dimensional channel systems have a system of pores intersecting, often in a mutually orthogonal manner, such that travel from any side of a zeolite crystal to another is possible. FAU is a three-dimensional zeolite comprising cages and pore openings delimited by 12-membered rings. LTL is a one-dimensional zeolite comprising pore openings delimited by 12-membered rings.

The catalyst may comprise a FAU zeolite. In another embodiment, the catalyst may comprise an LTL zeolite. The catalyst may additionally comprise potassium.

The catalyst may further comprise a binder, and be formulated into an extruded form. Shape of the catalyst may be a cylinder, trilobe, or other extrudable shape. Metal oxides may be preferred binders to provide mechanical strength and/or dilute a zeolite phase within the catalyst formulation. Catalyst formulations may be written in the form of Na-FAU/ZrO₂ or 75/25 Na-FAU/ZrO₂. This methodology of communicating the formulation may be read as zeolite comprising the catalyst formulation and oxide serving as the binder/diluent in the catalyst formulation. Numbers such as 75 and 25 should add up to 100 and show percentages of a given component in the formulation on a by weight basis.

The catalyst may comprise zeolite, one or more metal oxide sources, a source of potassium ions, a source of sodium ions, and combinations thereof. In one embodiment, the catalyst formulation may comprise between about 5% and about 95% FAU zeolite (e.g., CBV-100). The catalyst may comprise greater than about 25 wt % zeolite or greater than about 50 wt % zeolite or greater than about 70 wt % zeolite or greater than about 75 wt % zeolite. The catalyst may comprise less than about 95 wt % zeolite or less than about 90 wt % zeolite, or less than about 85 wt % zeolite. The catalyst may comprise less than about 25 wt % metal oxide or less than about 30 wt % metal oxide or less than about 50 wt % metal oxide or less than about 75 wt % metal oxide.

The catalyst may comprise potassium. The catalyst may comprise sodium. The catalyst may comprise sodium and potassium. In one embodiment, the catalyst formulation may comprise about 75% sodium FAU zeolite (e.g., CBV-100), between about 5% and 20% silica (e.g., Hi-Sil 250), and between about 5% and 20% alkali silicate source. Alkali silicate sources may comprise sodium silicate or may comprise potassium silicate. Alkali silicate sources may be selected from one or more of sodium silicate, potassium silicate, mixtures of sodium hydroxide and silica, mixtures of potassium hydroxide and silica, and combinations thereof. In one embodiment, the catalyst may comprise between about 5% and about 75% silica (e.g., Hi-Sil 250) and/or between about 5% and about 60% alkali silicate source. In one embodiment, potassium in the catalyst formulation may be present from one or more of ion-exchanged zeolite, metal oxide and/or binder sources, added separately, or a combination thereof.

The catalyst may possess a silicon to aluminum molar ratio (Si/Al) of greater than 2.0 or greater than 2.5 or greater than 3.5 or greater than 3.65 or less than 20.0 or less than 15.0 or less than 10.0 or less than 5.0. In an embodiment, the catalyst may possess a Si/Al of greater than 2.5 and less than 7.0.

The catalyst may possess a sodium to aluminum molar ratio (Na/Al) of greater than 0.5 or greater than 0.75 or greater than about 0.95 or less than 1.7 or less than 1.5 or less than about 1.35 or less than about 1.1. In an embodiment, the catalyst may possess a Na/Al of greater than 0.95 and less than 1.65.

The catalyst may possess a potassium to aluminum molar ratio (K/Al) of greater than about 0.01 or greater than about 0.15 or greater than about 0.20 or greater than about 0.25 or less than 1.0 or less than 0.75 or less than 0.50. In an embodiment, the catalyst may possess a K/Al of greater than 0.18 and less than 0.38.

The total cation to aluminum ratio may be determined by adding the Na/Al and K/Al values together. The catalyst may possess a total cation to aluminum ratio greater than about 1.10 or greater than about 1.20 or greater than about 1.30 and less than about 1.50 or less than about 1.45 or less than about 1.38. The catalyst may possess a nitrogen to aluminum ratio or N/Al ratio of less than 0.05 or less than 0.02, or about 0.

The process may further comprise contacting the catalyst with a second feedstream comprising an alcohol. The second feedstream may be combined with the first feedstream into a combined feedstream, or may be fed separately. Thus, the feedstream may comprise a lactic source and an alcohol. The alcohol may comprise one or more of methanol, ethanol, butanol, or mixtures thereof.

The feed stream may be converted into a product stream over the catalyst. The product stream may comprise one or more of acrylic acid, alkyl acrylate, a cation-balanced acrylate, or combinations thereof. The product stream may comprise one or more of acrylic acid, an acrylate balanced by a Group I cation or ammonium, an acrylate balanced by a Group II cation, an alkyl acrylate, or a combination thereof. The Group I cation may comprise one or more of lithium, sodium, potassium, cesium, or combinations thereof. The Group II cation may comprise one or more of magnesium, calcium, strontium, barium, or combinations thereof. The alkyl group may comprise a C₁-C₄ alkyl group such as but not limited to, for example, one or more of methyl, ethyl, isopropyl, and butyl. The alkyl acrylate may comprise one or more of methyl acrylate, ethyl acrylate, butyl acrylate, or combinations thereof.

Contacting of the feedstream with the catalyst to form a product stream may occur at a temperature of from about 200° C. or from about 250° C. or from about 280° C. to about 300° C. or to about 325° C. or to about 350° C. These temperatures reflect temperatures that are high enough to obtain conversion of the lactic source, but low enough that side reactions don't become the dominant pathway. Contacting of the feedstream with the catalyst may occur at a weighted hourly space velocity (WHSV) of from about 0.1 h⁻¹ to about 10 h⁻¹. The WHSV may be greater than about 0.1 h⁻¹ or greater than about 0.2 h⁻¹ or greater than about 0.4 h⁻¹ or greater than about 0.75 h⁻¹ or greater than about 1.2 h⁻¹ or less than about 8.0 h⁻¹ or less than about 5.0 h⁻¹ or less than about 2.0 h⁻¹ or less than about 1.2 h⁻¹.

Conversion of the lactic source during reaction may be greater than about 50% or greater than about 75% or greater than about 95% on a molar basis. Selectivity of the lactic source to dehydration products may be greater than about 50 mol %, or greater than about 75 mol % or greater than about 85 mol % or greater than about 90 mol %. Selectivity of the lactic source to acrylic products may be greater than about 50 mol %, or greater than about 75 mol % or greater than about 85 mol % or greater than about 90 mol %.

A further step in the process may comprise recovering the one or more of acrylic acid, alkyl acrylate, cation-balanced acrylate, or combinations thereof from the product stream. Recovering acrylic product may comprise one or more of liquid-liquid separation, distillation, membrane contactors, other known separation techniques, and combinations thereof. The product stream may be depressurized to a pressure lower than the reaction pressure prior to recovering the acrylic product.

### EXAMPLES

The following sets forth an order of tests performed and processes, tasks, or steps, relied on to support potassium containing catalyst formulations as a suitable alternative to adding an amine to a catalyst formulation. Specific formulations are provided by the tests and examples but are not meant to limit the present disclosure thereto.

### Example 1

Zeolite and metal oxide powders from commercial vendors were used as received for reaction activity measurements and catalyst formulations. Key characteristics of zeolite starting materials are summarized in Table 1. All chemicals, namely, methyl lactate [98%], ethyl lactate [98%], acetaldehyde [99.5%], methanol [99.9%], methyl acrylate [99%], ethanol [99%], ethyl acrylate [99%], 2,3-pentanedione [97%], methyl pyruvate [95%], acrylic acid [99%], 1,2-bis(4-pyridyl) ethane (12BPE) [99%], and 4,4-trimethylenedipyridine (44TMPD) [98%] were purchased from Sigma Aldrich, Spectrum Chemicals or Fisher Scientific and were used as received. Reverse osmosis water was used in all feed, reagent, and catalyst preparations, as needed.

TABLE 1
Table of ICP Values and subsequent calculated values.

| | Al2O3 | K2O | Na2O | SiO2 | Si/Al | Na/Al | K/Al | |
|---|---|---|---|---|---|---|---|---|
| Sample | Wt % | Wt % | Wt % | Wt % | (mole) | (mole) | (mole) | Charge/Al |
| Zeolyst Na-FAU (CBV- | 16.3 | <0.0585 | 10.1 | 49.5 | 2.80 | 1.00 | 0.00 | 1.00 |
| 100) | | | | | | | | |
| K-FAU (1X K exchange) | 18.6 | 15.1 | 3.69 | 57.1 | 2.56 | 0.32 | 0.86 | 1.18 |
| K-FAU (2X K exchange) | 18.3 | 14.4 | 3.61 | 56.0 | 2.55 | 0.32 | 0.84 | 1.16 |
| K-FAU (5X K exchange) | 18.2 | 17.7 | 0.143 | 55.6 | 2.55 | 0.01 | 1.03 | 1.05 |
| K-LTL | | | | | | | | |

### Example 2

Catalyst formulations containing mixtures of zeolite, metal oxide binder, and/or amine were prepared on a dry-weight basis. Formulations may further comprise small amounts (<5%) of extrusion aids such as cornstarch or Methocel (Dow). Loss on ignition (LOI) of solid materials was determined by heating to 550° C. for 3 hours to calculate the equivalent wet-weight required to obtain a desired dry-weight mass ratio. Extrusion aids generally combust prior to about 550° C. and generally do not contribute to mass of the final extruded catalyst formulation.

Most extrudates were produced on a 5 to 15-gram lab scale using a handheld extruder. After mixing, the dough was extruded through a ⅛″ diameter die plate in a hand-held extruder and allowed to air dry prior to calcination at 550° C. for 3 hours. Extruded pellets were crushed and sieved prior to reaction activity measurements. Percentages are based on weight (i.e. dry weight) remaining after loss on ignition (LOI) determination at 550° C. for 3 hours. For milligram-scale testing using microreactor Plant A, pellets were sieved to 25 to 60 mesh whereas for gram-scale testing using Plant B, pellets were sieved to 10 to 25 mesh.

Formulations given here in Table 2 were prepared with various percentages of Zeolyst Na-FAU CBV-100 as zeolite starting material, Hi-Sil 250 silica and Thermo Fisher potassium silicate starting materials unless stated otherwise in the following paragraph. All percentages must add to 100 percent. Variations in Si/Al, Na/Al, and K/Al are achieved by varying percentages of the starting materials.

TABLE 2
Extruded Samples

| | FAU | | | | | XRD | XRD peak |
|---|---|---|---|---|---|---|---|
| Formulation | content (%) | Si/Al | Na/Al | K/Al | Scale | Crystallinity | ratio |
| 20 | 74 | 3.81 | 1.45 | 0.01 | Lab | 71% | 1.43 |
| 21 | 50 | 6.40 | 1.66 | 0.01 | Lab | 52% | |
| 32 | 75 | 4.00 | 1.60 | 0.01 | Lab | 83% | |
| 35 | 74 | 3.84 | 1.05 | 0.28 | Lab | 83% | 1.34 |
| 36 | 74 | 4.01 | 0.47 | 1.34 | Lab | 66% | |
| 48 | 74 | N/D | N/D | N/D | Lab | N/D | |
| 56 | 50 | 6.40 | 1.15 | 0.81 | Lab | 49% | 1.09 |
| 59 | 25 | 14.01 | 1.37 | 2.44 | Lab | 27% | |
| 57 | 0 | Infinity | Infinity | Infinity | Lab | N/D | |
| 60 | 74 | 3.71 | 1.05 | 0.41 | Lab | 75% | 1.25 |
| 61 | 74 | 3.82 | 1.03 | 0.09 | Lab | 79% | |
| 62 | 25 | 13.61 | 1.47 | 4.03 | Lab | 18% | |
| 63 | 25 | 14.22 | 1.31 | 0.94 | Lab | 25% | |
| 65 | 25 | 14.01 | 4.55 | 1.52 | Lab | 7% | |
| 66 | 76 | 3.85 | 1.33 | 0.15 | Lab | 71% | 1.29 |
| 69 | 74 | 3.67 | 1.03 | 0.25 | Lab | 76% | |
| 70 | 74 | N/D | N/D | N/D | Lab | 71% | |
| 71 | 75 | 3.68 | 1.06 | 0.24 | Lab | N/D | |
| 74 | 75 | 3.68 | 1.06 | 0.24 | Lab | 81% | 1.42 |
| 75B | 74 | 3.84 | 1.03 | 0.29 | 350 g | 81% | 1.40 |
| 75C | 74 | 3.82 | 1.03 | 0.29 | 350 g | 78% | |
| 75D | 74 | 3.74 | 1.01 | 0.28 | 350 g | 81% | |
| 84 | 75 | 3.79 | 1.07 | 0.24 | Lab | 82% | |
| 85 | 75 | 3.90 | 1.09 | 0.25 | Lab | 82% | |
| 87 | 75 | 4.01 | 1.07 | 0.30 | 750 g | 72% | 1.25 |
| 90 | 72 | 4.08 | 1.10 | 0.31 | Lab | 79% | 1.28 |

Formulation 36 was produced with the same percentages as Formulation 35, by using K⁺-FAU zeolite (ion-exchanged CBV-100, Example 3) in the formulation in place of Na-FAU. Formulations 65 and 66 further comprise sodium silicate (Sigma-Aldrich). Catalyst formulations 20, 21, and 32 comprise sodium silicate in place of potassium silicate. Formulations 69 and 70 are the same as Formulation 35 but utilize Hi-Sil 532EP silica (PPG) and Hi-Sil EZ200G (PPG) respectively in the formulation in place of Hi-Sil 250.

The kg scale Rondol 10 mm co-rotating twin-screw 25 L/D kg scale extruder has a continuous powder feeder and a continuous liquid feeder. The mixture of components for the powder feed into the extruder are measured and reported based on dry weight. A target mass of powder feed to be made is chosen, and the required masses of each component are then calculated, first on a “dry” basis then on a “wet” basis, as they are received out of the bottle. The components are weighed out and mixed in a large plastic beaker. The flow rate of powder from the powder feeder is based on the RPM setting of the powder feeder. The mass flow rate of the powder therefore depends on the density of the powder, and other less easily measured powder rheology characteristics. To determine the relationship between powder feeder RPM and mass flow of powder, a calibration must be performed on each different powder feed. Water was fed via the liquid feed peristaltic pump to a target dough LOI.

Formulations 75B, 75C, and 75D were produced by altering feed rates from a single combined feed powder that is the same formulation as 35 although 3.0 g of Methocel F50 (Dow) was additionally present in the 360 g of powder. Extrusion was carried out for some time. All formulations use 250 rpm screw rate. 75B uses 40 rpm powder feed and 10.7 rpm H₂O feed. 75C uses 50 rpm powder feed and 12.5 rpm H₂O feed. 75D uses 50 rpm powder feed and 12.5 rpm H₂O feed. Extrudates were collected, dried, and then calcined in air to yield 60.96 g total catalyst formulation 75. Solids in the powder feeder were not fully consumed in this particular example. Formulations 35, 74, 75, 84, 85, and 87 utilize the same powder formulation, but with extrusion at different scales and extrudate water content. Each formulation performs equivalently within error of repeat runs on the plant.

### Comparative Example 3

Additional comparative catalyst formulations were prepared. The catalyst formulation 13 was produced by extruding 75% CBV-100 with 25% silica (Hi-Sil 250) in a hand extruder. This formulation was also scaled up to 350 g batch size by extrusion in a kg scale lab extruder (Formulation 38). The catalyst formulation 4 was produced by extruding 75% CBV-100 with 25% alumina (Al(OH)₃, Sigma-Aldrich) in a hand extruder. The catalyst formulation 12 was produced by extruding 50% CBV-100 with 50% kaolinite clay (Sigma-Aldrich) in a hand extruder. Catalyst formulations 18 and 19 were prepared by extruding 75% and 50% CBV-100 respectively with bentonite clay.

TABLE 3
Extruded Samples

| | FAU | | | | | XRD |
|---|---|---|---|---|---|---|
| Formulation | content (%) | Si/Al | Na/Al | K/Al | Scale | Crystallinity |
| 4 | 75 | N/D | N/D | N/D | Lab | 77% |
| 12 | 50 | N/D | N/D | N/D | Lab | 50% |
| 13 | 75 | 4.01 | 1.05 | 0.01 | Lab | 77% |
| 18 | 75 | 2.52 | 0.79 | 0.01 | Lab | 77% |
| 19 | 50 | 2.59 | 0.58 | 0.01 | Lab | 51% |
| 38 | 75 | N/D | N/D | N/D | Kg | 72% |

TABLE 4
Composition of solids used in Formulation 38

| | | Dry mass | Wet mass | Actual |
|---|---|---|---|---|
| Component | LoI | eq (g) | target (g) | mass |
| CBV-100 | 22.8% | 178.01 | 230.58 | 230.39 |
| Hi-Sil 250 | 14.6% | 59.34 | 69.48 | 69.51 |
| Methocel | — | — | 3.00 | 3.01 |

### Example 4

Potassium ion exchanged zeolites (Z) were prepared by ion exchange of their corresponding M-Z sample in an aqueous solution of 1 M KNO₃ (10 mL per dry-weight gram of zeolite) at 80 to 90° C. for 1 hour. The resultant K-Z zeolites were then filtered, dried at 110° C. overnight, and calcined at 550° C. for 3 hours. This procedure was repeated sequentially to obtain zeolite samples potassium ion exchanged to greater extents. Analytical results are shown in Table 1. Extrudate formulations may also be ion-exchanged. Catalyst formulation 48 was synthesized by ion-exchanging formulation 20 using the procedure above.

### Comparative Example 5

Multifunctional amines were loaded onto catalysts using wet impregnation or milling during extrusion. In the case of wet impregnation, zeolite powder or extruded zeolite/binder pellets were added to a methanol solution containing dissolved amine and stirred for 4 hours. The slurries were then dried at 70° C. overnight to yield the amine-impregnated catalyst. In the case of milling, a certain mass of amine solid was added to the wet dough mixture of zeolite, binder(s), and extrusion aid. The components were mixed and water was added to generate an extrudable dough, after which it was extruded and air-dried. Notably, extrudates containing amine incorporated via impregnation or milling were calcined at 300° C. rather than 550° C., as the elevated temperature would destroy the amine additive. For all impregnations and milling, amines were added at a 25 weight % loading relative to the dry weight of the zeolite in the final formulation.

### Example 6

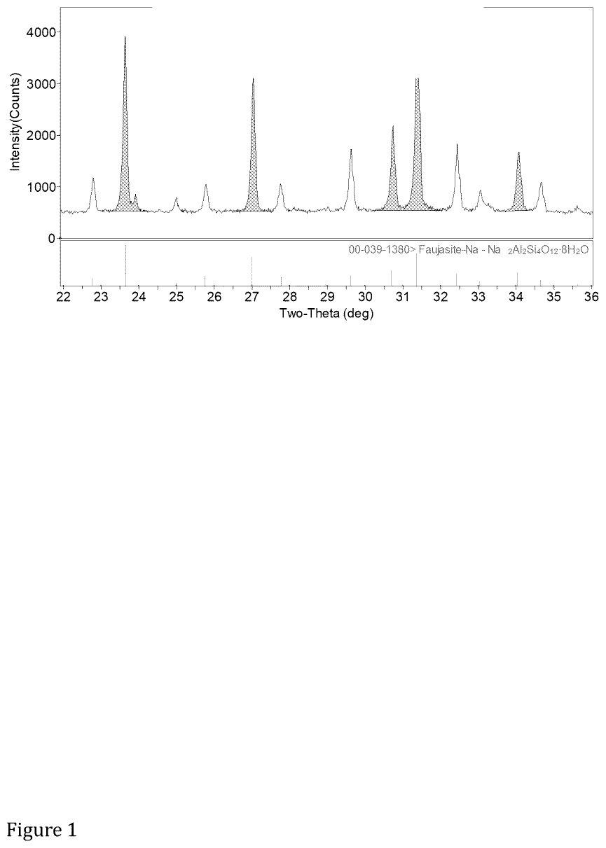

Powder X-ray diffraction was carried out on a Proto AXRD powder diffractometer equipped with a cobalt source. Qualitative diffraction scans were performed from 3°-70° Two Theta (cobalt)/2.6°-59° Two Theta (copper). Quantitative scans for integration of the FAU zeolite content were run from 26°-41.5° (cobalt). Six peaks in this range were integrated (533), (622), (642), (822), (555) & (644) (FIG. 1). The total integrated intensity was corrected for tube decay and sample x-ray absorption effects and compared to the integrated intensity of a FAU standard zeolite (CBV-100) to determine XRD Crystallinity of the FAU catalyst component. Similar measurement can be performed for LTL or other zeolite structures. Alpha-alumina was used as a daily standard to correct for tube decay. Mass Adsorption Calculations were used to correct for components with high absorption which may have been added in the catalyst forming process. These calculations were performed based on sample elemental composition combined with X-ray mass attenuation coefficients published by NIST. Elemental analysis was performed by a contract analytical laboratory. % Crystallinity values determined by XRD can be found in Tables 2 and 3.

TABLE 5
Table of commercial zeolites used in further experiments with key characteristics.
XRD crystallinity is normalized relative to Zeolyst CBV-100 Na-FAU.

| | Structure | | | Cation | Si/Al | XRD |
|---|---|---|---|---|---|---|
| Entry | Code | Supplier | Supplier Code | Type | ratio | crystallinity |
| 1 | FAU | Zeolyst | CBV-100 | Na | 2.6 | 100% |
| 2 | FAU | Zeolyst | CBV-500 | NH₄ | 2.6 | 83% |
| 3 | FAU | Zeolyst | CBV-712 | NH₄ | 6 | 75% |
| 4 | FAU | Tosoh | HSZ320NAA | Na | 2.85 | 48% |
| 5 | FAU | Zeolyfe | ZB040 8-21 | Na | | 96% |
| 6 | FAU | Sigma | 208639 | Na | 1.2 | 85% |
| 7 | LTL | Tosoh | HSZ500KOA | K | 3.1 | |
| 8 | MOR | Tosoh | 642NAA | Na | 9 | |
| 9 | MFI | Tosoh | HSZ-890HOA | H | 885 | |
| 10 | MFI | Tosoh | HSZ-820NHA | NH₄ | 11.7 | |

### Example 7

Reaction activity measurements were primarily performed at atmospheric pressure and 300° C. in a packed bed microreactor contained in a modified Agilent 8890 gas chromatograph (GC). This microreactor is referred to as Plant A and is modeled after the “catalyst-in-a-box” testing system developed by the Dauenhauer Lab at the University of Minnesota and described in previous work. Milligram scale catalyst samples were sandwiched between two layers of deactivated quartz wool in an Agilent GC inlet liner. Typical bed dimensions were 4-mm dia. X 1-cm height. Methyl or ethyl lactate was fed to a vaporizer upstream of the reactor tube by a syringe pump via a 1/16″ O.D.×0.002″ I.D. polyetheretherketone (PEEK) tube. Unless otherwise stated in a particular experiment, the feed used was 30 wt % methyl lactate in water. Vaporized alkyl lactate feed flowed through the catalyst bed, with reactor effluent then flowing through a GC sampling valve to permit real-time analysis of product stream composition via GC separation and quantitation. Reactor effluent was separated by an Agilent HP-FFAP column (PN 19091F-112) and analyzed via a quantitative carbon detector (QCD, Polyarc) in series with a flame ionization detector (FID).

Larger scale catalyst testing was conducted in a laboratory-scale plant, for quantification of feed and reaction products from a traditional fixed bed gas-phase flow reactor for heterogeneous catalyst screening at 1-20 g scale. Plant B was constructed to evaluate a wider range of operating conditions, including feed and diluent gas flow rates, reaction temperature and pressure, diluent gas type, and run time. Plant B was also equipped with an on-line Agilent 8890 GC for real-time analysis of reactor effluent. Reactor effluent was separated by an Agilent HP-FFAP column (PN 19091F-112) and analyzed via a quantitative carbon detector (QCD, Polyarc) in series with a flame ionization detector (FID).

For the purposes of high throughput reaction activity screening, the weighted hourly space velocity (WHSV) was kept at a constant value of 0.4±0.05 h⁻¹ for all testing on Plant A and B. Weighted hourly space velocity was defined as:

$$W H S V = \frac{mass flowrate of reactant}{dry weight equivalent mass of zeolite in catalyst}$$

Feed concentration was fixed at 30 weight % alkyl lactate in an aqueous solution while the catalyst loading and feed flowrate were varied to maintain a constant WHSV value of 0.4 h⁻¹. Reaction activity measurements on Plant A required catalyst loads between 50 and 180 mg and feed flow rates of 1 μL/min, while Plant B required catalyst loads between 1 and 5 g and feed flow rates between 20 and 100 μL/min. Prior to catalytic testing, catalyst samples were calcined at 300° C. for 5 hours in one of two conditions; 1) ex-situ in an isothermal heating block under an ambient atmosphere or 2) in-situ in the plant under continuous nitrogen gas flow.

### Example 8

To evaluate the impact of varying zeolite structure type and characteristics on the lactic-to-acrylics reaction, a variety of commercially available zeolites detailed in Examples 1 and 6 were tested in the high-throughput microreactor Plant A (Table 2). Zeolites tested included variations of the base faujasite structure (“FAU”) as well as zeolites belonging to different zeotypes.

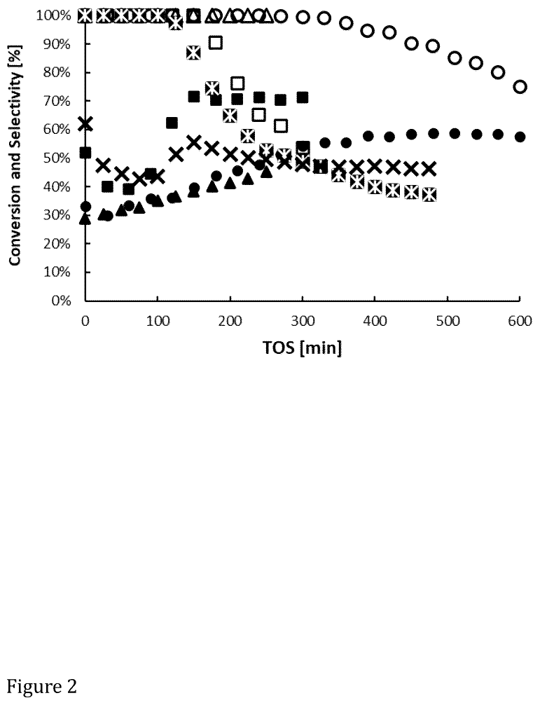

First, Na-FAU zeolites from four different commercial manufacturers were tested to assess the impact of vendor source on catalytic performance (FIG. 2). Conversion is shown in open symbols, and dehydration selectivity shown in closed symbols. Na-FAU zeolites from Zeolyst (circles out to 600 min TOS), Tosoh (triangles to 300 min), Zeolyfe (triangles out to 300 min TOS) and Sigma Aldrich 13× molecular sieves (dark box with hash mark is conversion, x markers are selectivity out to 500 min TOS) are shown. Tosoh Na-FAU achieved 100% conversion of methyl lactate for 250-minutes time on stream (TOS). Dehydration selectivity rose with TOS, reaching a maximum of 45% at 250-min. Zeolyst Na-FAU yielded similar performance as Tosoh Na-FAU, with conversion gradually decreasing from 100% after 300-min TOS and selectivity rising with TOS to a maximum of ˜55% after 500-min (FIG. 2a). Zeolyfe Na-FAU exhibited a different conversion profile compared to either Tosoh or Zeolyst, maintaining 100% conversion of methyl lactate for only 150-min TOS before deactivating rapidly (FIG. 2b). However, dehydration selectivity reached a greater maximum of 72% after 150-min TOS.

Commercial molecular sieves obtained from Sigma-Aldrich containing Na-FAU were also tested. 13× beads were crushed and sieved to 25-60 mesh before being tested under equivalent process conditions as other zeolite catalysts on Plant A. Methyl lactate conversion rapidly decreased from 100% after 150-min TOS for 13× beads, faster than Na-FAU powder from Zeolyst, Tosoh, or Zeolyfe (FIG. 2b). Dehydration selectivity reached a maximum of 50% after 150-min TOS before decreasing gradually (FIG. 2b).

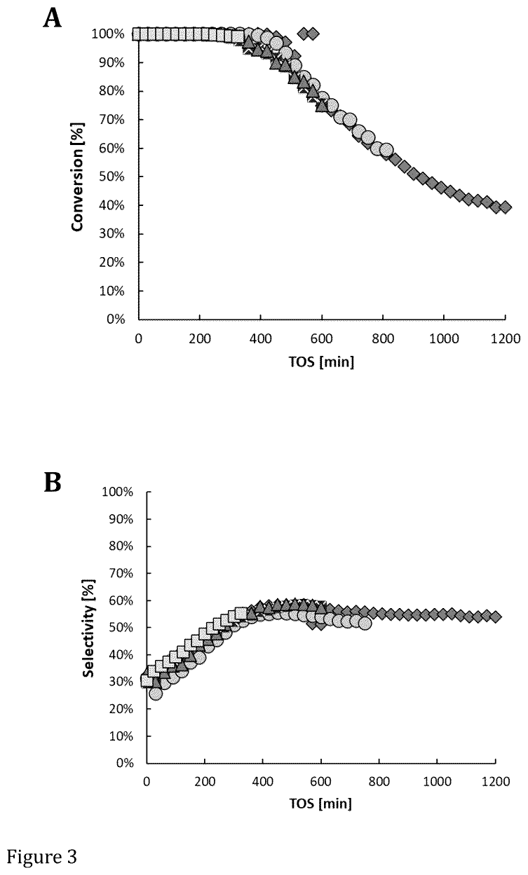

Five replicate runs were performed with Zeolyst Na-FAU under equivalent process conditions to evaluate the typical run-to-run variability characteristic of the Plant A microreactor. Zeolyst Na-FAU reliably achieved 100% conversion for 300 to 400-min TOS before deactivating gradually (FIG. 3a). Dehydration selectivity consistently reached a steady maximum of 55 to 60% after a 400-min TOS induction period (FIG. 3b). Each of the 5 runs shown with squares, circles, diamonds, triangles, and box/X overlay on top of each other.

Two additional FAU zeolites were tested containing ammonium (NH₄) as the predominant cation on the zeolite acid sites: Zeolyst NH₄-FAU with nominal silicon to aluminum ratios of 2.6 and 6. Methyl lactate achieved near 100% conversion for 300-min TOS for both NH₄-FAU zeolites. However, dehydration selectivity was at or near zero, with >95% of products comprised of COₓ, acetaldehyde, and methanol. The high activity of NH₄-FAU towards methyl lactate decarbonylation demonstrates the importance of the zeolite cation in determining selectivity towards dehydration. Clearly, NH₄ is not a strong enough cation; presumably NH₃ is driven off, leaving the Brønsted acid catalytic site known to perform decarbonylation side reactions.

### Example 9

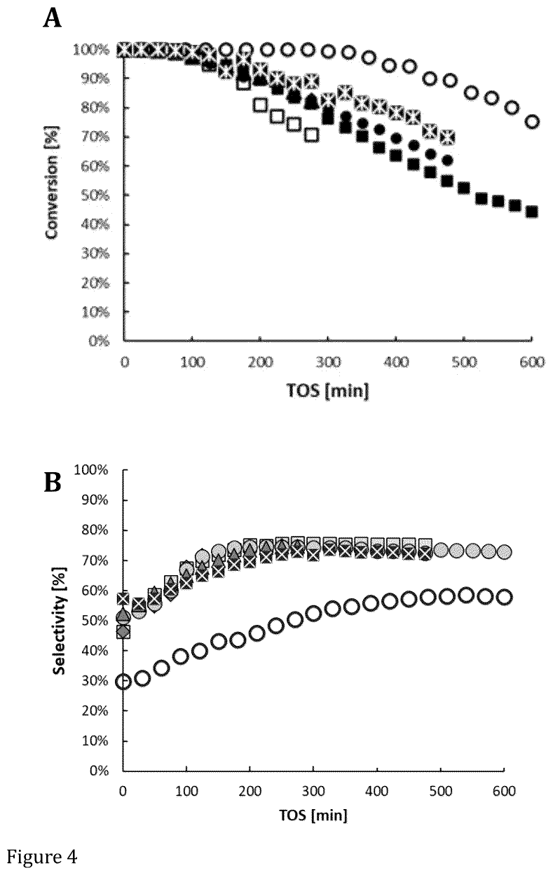

Potassium ion exchanged Zeolyst Na-FAU Materials from Example 4 afforded a significant increase in dehydration selectivity. Unfortunately, catalyst lifetime during testing decreased compared to the base Na-FAU material (FIG. 4a). The number of successive K⁺ ion exchanges did not have an impact on dehydration selectivity; rather, all levels of ion exchange yielded the same maximum selectivity of ˜75% after a 200-min induction period (FIG. 4b) with no correlation to catalyst lifetime even as Na⁺ was removed in favor of K⁺ (Table 1).

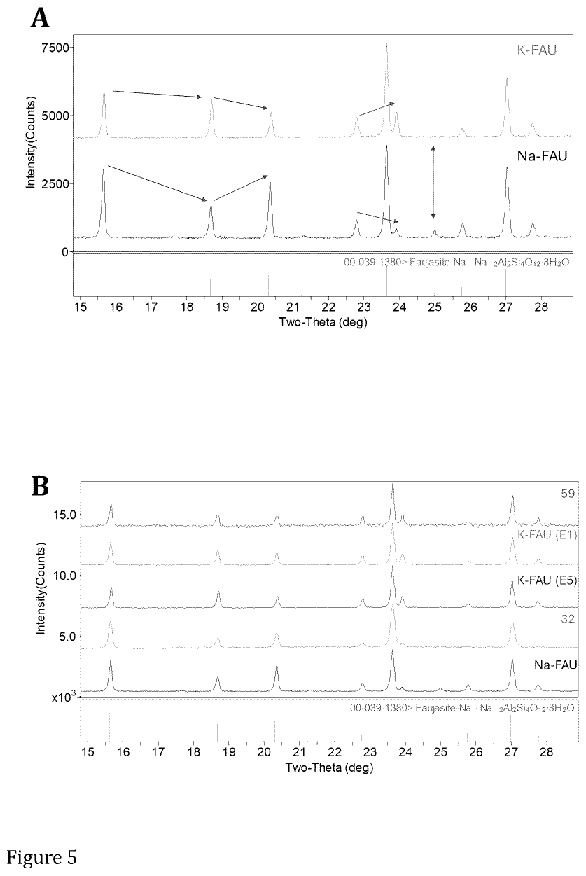

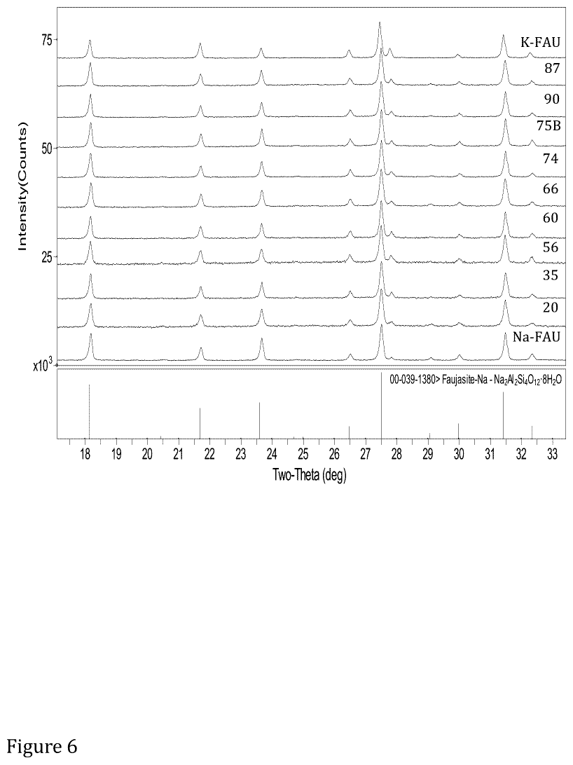

These results may be correlated to observed differences in XRD patterns between FAU material as a function of the alkali cation. As shown in FIG. 5A, the (444) reflection of Na-FAU at 24.8° 2θ disappears upon ion exchange to K-FAU. At 1× ion-exchange, the (444) reflection has mostly disappeared. Catalysts of the invention such as Formulation 59 have little intensity at this location. Peak ratios throughout the XRD pattern also change; this is readily visible in FIG. 5A for the 3 peaks between 15° and 21° 2θ and pointed out by arrows. Peak ratios also change rapidly upon K⁺ inclusion towards that of K-FAU (FIG. 5B and FIG. 6) for catalysts of the instant application. While the extent of Na⁺ substitution for K⁺ was impacted by the number of ion exchanges (Table 1), these XRD patterns suggest that degree of K⁺ incorporation results in a discrete and uniform change in FAU structure. In particular, for K+-FAU, the peak height of the FAU (440) reflection at 20.4° 2θ (copper radiation) is less than the height of the FAU (511) reflection at 18.7° 2θ (copper radiation) for catalysts of the application. FIG. 6 shows additional XRD patterns of formulations from Table 2. The ratio of the peak height of the (440) reflection can be divided by that of the (551) reflection to yield the XRD peak ratio. For example, the Zeolyst Na-FAU starting material has an XRD peak ratio of 1.74 (3946 peak height divided by 2267 peak height) and the fully ion-exchanged K-FAU material has an XRD peak ratio of 0.67 (1584 peak height divided by 2364 peak height). The XRD peak ratio for catalysts of the application may be greater than 1.00 or greater than about 1.10 or greater than about 1.15 or greater than about 1.20 and less than about 1.65 or less than about 1.55 or less than about 1.50.

### Example 10

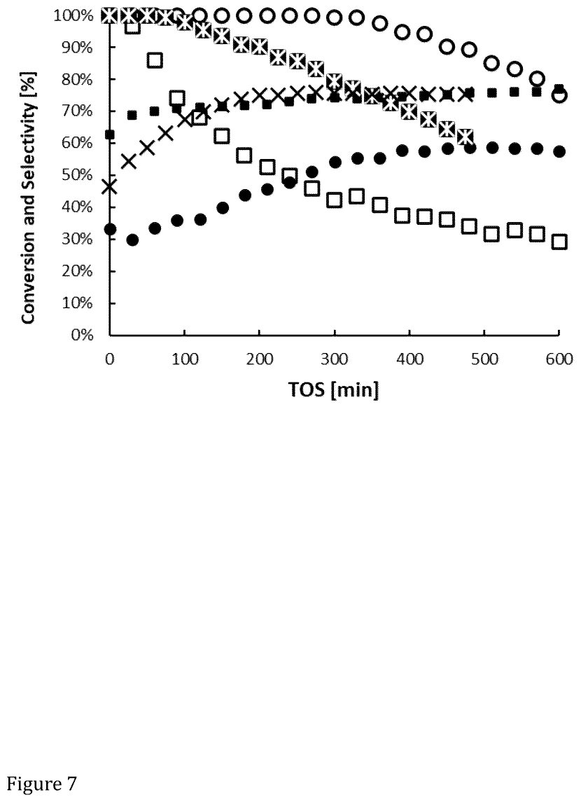

Potassium form Linde type L zeolite (“K-LTL”) from Example 1 and characterized in Example 5 was additionally tested to further probe the importance of the cation identity under conditions of varying crystallite structure. Plot of conversion and dehydration selectivity is shown in FIG. 7, where K-FAU (dark box with hash mark is conversion, x markers are selectivity) and K-LTL (squares) compared to the performance of base Na-FAU zeolite (circles) and conversion is open symbols, dehydration selectivity closed. Conversion of methyl lactate decreased rapidly from 100% immediately upon starting the reaction for K-LTL, faster than either K-FAU or Na-FAU (FIG. 7). However, K-LTL achieved dehydration selectivity comparable to K-FAU, reaching a maximum of 79% over 600-min TOS. Both potassium-containing zeolites (K-LTL and K-FAU) achieved superior dehydration selectivity relative to sodium form FAU. However, deactivation of K-LTL and K-FAU was more rapid than Na-FAU.

### Example 11

Sodium form mordenite zeolite (Na-MOR) from Example 1 and characterized in Example 5 exhibited poor conversion and dehydration selectivity compared to sodium form FAU. Both methyl lactate conversion and dehydration selectivity were near or below 10% over 600-min TOS.

A single potassium ion exchange (Example 3) of the proton or ammonium form MFI zeolite (Examples 1 and 5) yielded enhanced dehydration selectivity but worse methyl lactate conversion. For K/H-MFI, dehydration selectivity increased from zero to 30% while conversion dropped from 100% to 20%. For K/NH₄-MFI, dehydration selectivity increased from near-zero to nearly 60% after 1000-min TOS while conversion dropped from 100% to 8%.

### Comparative Example 12

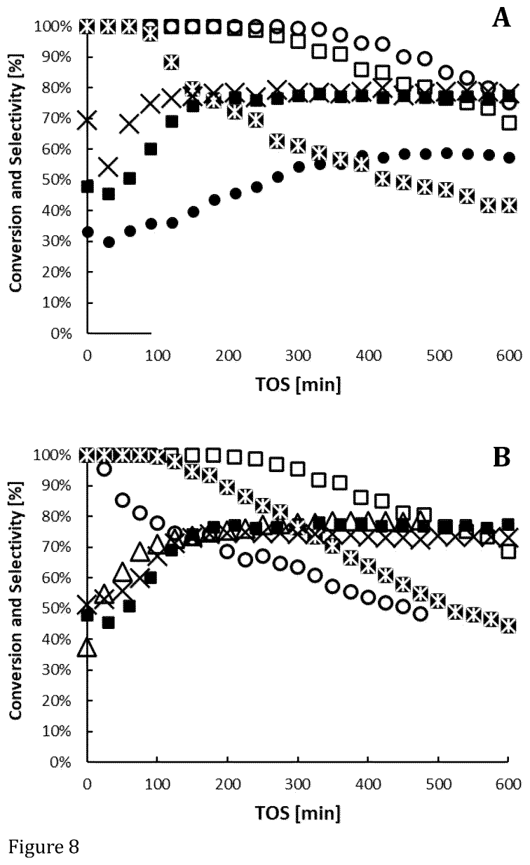

Impregnation of Na-FAU with multifunctional amines (Example 4) afforded enhanced dehydration selectivity due to the suppression of the undesirable decarbonylation pathway. Na-FAU impregnated with either 4,4′-trimethylenedipyridine (44TMDP) or 1,2-Bis(4-pyridyl) ethane (12BPE) at a nominal 25-wt % loading achieved dehydration selectivity of 80% after a 300-min induction period, compared to selectivity of ˜55% for standard Na-FAU (FIG. 8). However, both 44TMDP and 12BPE impregnated Na-FAU had reduced catalyst life versus base Na-FAU. Methyl lactate conversion of amine-impregnated Na-FAU dropped from ˜100% upon starting the reaction, while standard Na-FAU maintains full conversion for 300 to 400-min TOS (FIG. 8).

### Example 13

Scale-up of catalyst formulation had been investigated through the preparation and testing of extruded zeolite catalysts (Example 2). On a commercial scale, catalyst pellets are used in fixed-bed reactors, not powders. Packed (or immobilized) pellets allow for fluid flow through the reactor while still maintaining high surface area for contact with reactants. Pellets must have the strength to withstand the force of their own weight within the reactor. An inert catalyst support, or binder, is utilized to provide sufficient strength and to enable efficient catalyst extrusion. FIG. 8 shows catalytic dehydration of methyl lactate to acrylics over several catalysts. Part A shows Formulation 35 (red circles) and Formulation 20 (blue circles) against Na-FAU. In comparison, Part B shows long lifetime achieved over Formulation 35 (red circles) at the same selectivity as 44TMPD impregnated FAU from Example 5 (yellow) and K-FAU from Example 4 (blue).

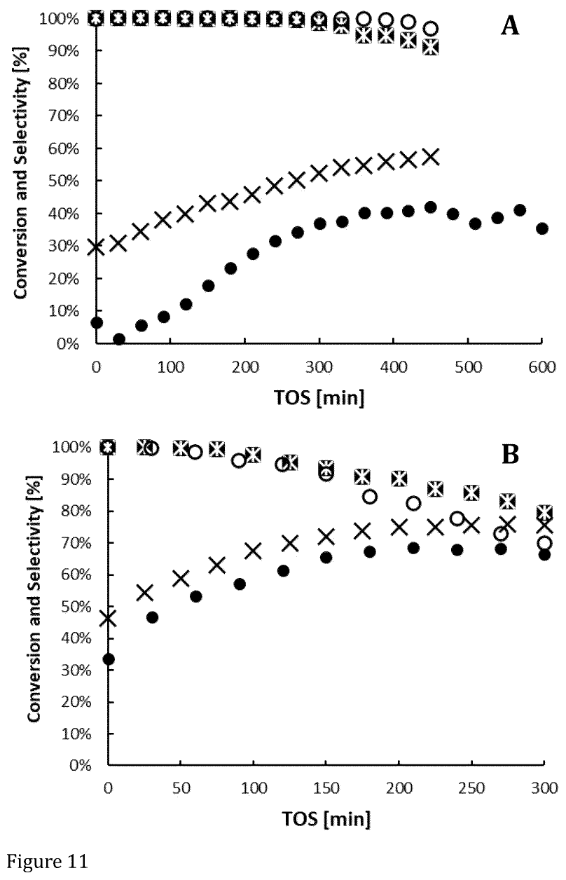

Multiple binders were explored using Zeolyst Na-FAU as the base zeolite due to product availability, feasibility at commercial scale, and strong performance versus alternative zeolites. A Na-FAU formulation containing a silica-based binder yielded surprising catalytic performance, with conversion and selectivity profiles exceeding plain Na-FAU and alternative formulations. This work was extended to further investigate silica-containing binders. The significance of the binder as an alkali cation source was revealed. Additional silica binders were investigated, with two containing alkali cations exhibiting significantly enhanced performance versus the base Na-FAU material. Formulations 20 and 35 (Example 2, FIG. 8) achieved dehydration selectivity of over 80% compared to the 55% selectivity achieved by base Na-FAU (FIG. 11 (a)). While methyl lactate conversion decreased for Formulation 20 (FIG. 8 blue series), the catalyst life of Formulation 35 was comparable to the base Na-FAU starting material (FIG. 8, red and black series).

This inventive Formulation 35 (Example 2) exhibited a high dehydration selectivity of over 80%, which was similar to other high-performing FAU catalysts tested by Låkril technologies under these process conditions, including K-FAU (Example 9) and 44TMDP amine impregnated Na-FAU (Comparative Example 12) as shown in (FIG. 8). However, the Formulation 35 catalyst additionally exhibited a greater life than K-FAU or 44TMDP amine impregnated Na-FAU (FIG. 8). Incorporation of the alkali binder resulted in a 25% enhancement in dehydration selectivity.

### Example 14

To further evaluate the impact of the alkali cation on lactic acid dehydration, potassium ion exchanged derivatives of extruded Na-FAU formulations (i.e., Formulation 35 and Formulation 20) were prepared using the procedures of Examples 2 and 3.

First, an extruded FAU formulation analogous to Formula 20 was prepared using potassium-ion exchanged FAU powder (K-FAU) rather than the sodium form faujasite (Na-FAU). The K-FAU analog Formula 29 achieved dehydration selectivity comparable to the original Na-FAU based formulation (Formula 20) as well as to plain K-FAU powder. However, methyl lactate conversion was markedly lower and decayed more rapidly.

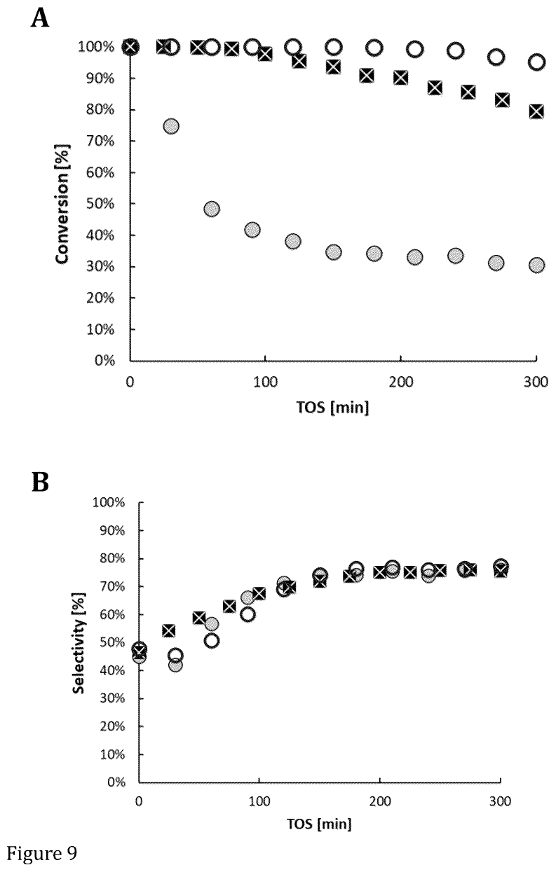

A potassium-form analog to Formula 35 was prepared using K-FAU powder rather than the Na-FAU powder in the original formulation. Just as with Formula 29, this potassium form analog (Formula 36) achieved worse methyl lactate conversion but comparable dehydration selectivity (FIG. 9). Total cation to Al ratio (Table 2) may be too high in Formula 36.

The formulation featuring K⁺ ion exchanged FAU powder (Formulation 36, gray circles) exhibited worse conversion but equivalent selectivity compared to base K-FAU zeolite (squares with X) or Formulation 35 open circles as shown in FIG. 9. So far, K-FAU analogs to the Formulation 20 and Formulation 35 yielded inferior catalytic performance compared to the base sodium form. In the case of potassium analogs making use of K-FAU powder as a raw material for the extruded catalyst, equivalent dehydration selectivity was achieved relative to either the Na-FAU form extrudate analog or K-FAU powder starting material. However, in all cases, K-FAU starting materials yielded significantly worse conversion profiles.

### Example 15

The amine 44TMDP was incorporated into the Na-FAU/silica catalyst RD-0317 via three different methods. First, an amine analog to Formula 20 was prepared using 44TMDP amine impregnated Na-FAU powder (Example 4) as the starting material instead of plain Na-FAU (Formula 28). A second amine analog was prepared by incorporating the solid 44TMDP amine into the wet extrusion dough at the same time as the zeolite, binders, and extrusion aid (Formula 30). A third amine analog was prepared by performing a wet impregnation of extruded pellets of Formula 20 using 44TMDP amine dissolved in methanol (Formulation 9). In all amine analogs, 44TMDP amine was impregnated at a 25 weight-% loading, and the zeolite contents were equivalent to the Formula 20 benchmark formulation.

For all three amine containing analogs of Formula 20, methyl lactate conversion decreased relative to either the Formula 20 benchmark catalyst or 44TMDP amine impregnated Na-FAU powder while dehydration selectivity remained unchanged at around 75%.

While potassium ion and 44TMDP amine incorporation generally decreased the catalytic performance of the Formula 20 benchmark formulation individually, additional analogs showed the impact of both the potassium and the amine treatments being added together. The first potassium/amine analog of Formula 20 was prepared using K-FAU powder as the starting material (Example 3) which was extruded with the silica binders per the standard formulation 20 recipe (Example 2). This potassium form extrudate was then impregnated with 44TMDP amine at a 25 weight-% loading to yield formulation 11. A second potassium/amine analog of Formulation 20 was prepared using the same K-FAU powder starting material as Formulation 11, but the 44TMDP amine was incorporated via mixing into the wet extrusion dough at the same time as the K-FAU zeolite, silica binders, and extrusion aid to form Formulation 33.

Both potassium/amine analogs exhibited inferior catalytic performance compared to Formulation 20 benchmark. Methyl lactate conversion for the analogs fell rapidly to near 20% over 600-min TOS, worse than Formulation 20 benchmark catalyst or Na-FAU powder raw material treated singly with either potassium or 44TMDP amine. Selectivity was lowest for the Formulation 33, reaching a maximum of 50% over 600-min.

Impact of the amine, cation identity, cation content and the binder on the properties of the formulated catalyst is recognized. The cation may comprise potassium. Further synthetic methods are required to manufacture catalysts with the appropriate proportions and spatial arrangement of zeolite, cation, binder, and amine. The differential impact of the amine impregnation on conversion as a function of binder choice (alumina, zirconia or silica) demonstrates that optimization of binder, cation, and amine incorporation is merited.

### Example 16

Performance of key catalysts in the lactic-to-acrylics reaction was evaluated using 30 wt % ethyl lactate (EL) as the lactic feed as opposed to methyl lactate (ML). Catalytic data was collected using the same milligram-scale microreactor (Plant A) under equivalent process conditions as used while testing with methyl lactate.

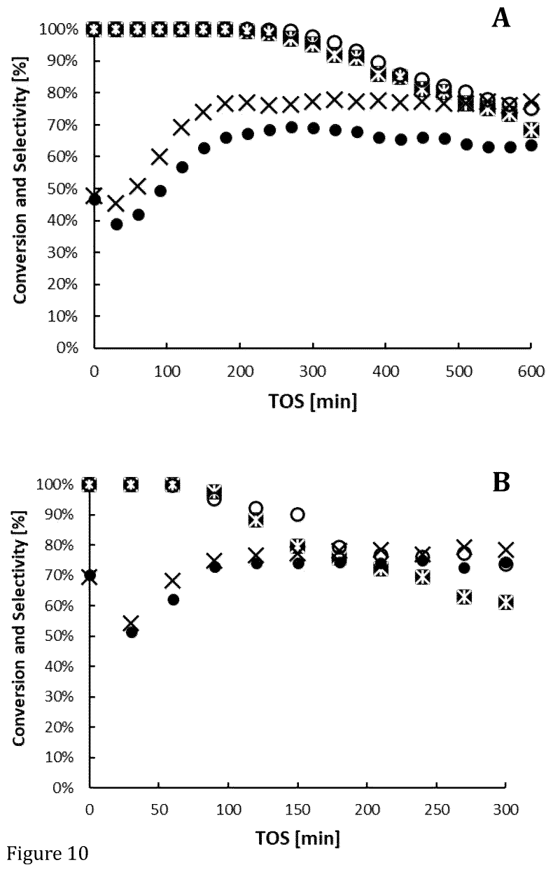

Formulation 35 containing K⁺ was tested twice using 30 wt % EL prepared using either Spectrum Chemicals or Sigma Aldrich EL. Alkyl lactate conversion and dehydration selectivity were equivalent for the two feedstock manufacturers based on the typical run-to-run variability demonstrated on Plant A with Na-FAU and ML feed (FIG. 3). Compared to catalytic performance using ML feed, Formulation 35 achieved equivalent alkyl lactate conversion but a reduction in maximum dehydration selectivity of around 10%, decreasing from 80% to 70% (FIG. 10). Without being bound by theory, it is believed the drop in selectivity is due to dehydration of ethanol produced during reaction to ethylene; from this, the competitive rate of dehydration of lactic acid versus ethanol in this system may be determined.

FIG. 10 shows catalytic dehydration of methyl lactate versus ethyl lactate over Formulation 35 (Part A) and Formulation 20 (Part B) as discussed in Example 16. In each part, conversion of methyl lactate is shown in open circles, conversion of ethyl lactate in closed circles, methyl lactate dehydration selectivity in open triangles, and ethyl lactate dehydration selectivity in closed triangles. Formulation 20 also achieved equivalent alkyl lactate conversion profiles under ML and EL feeds. However, dehydration selectivity was also lower with EL, reaching a maximum of 73% for EL compared to 80% for ML.

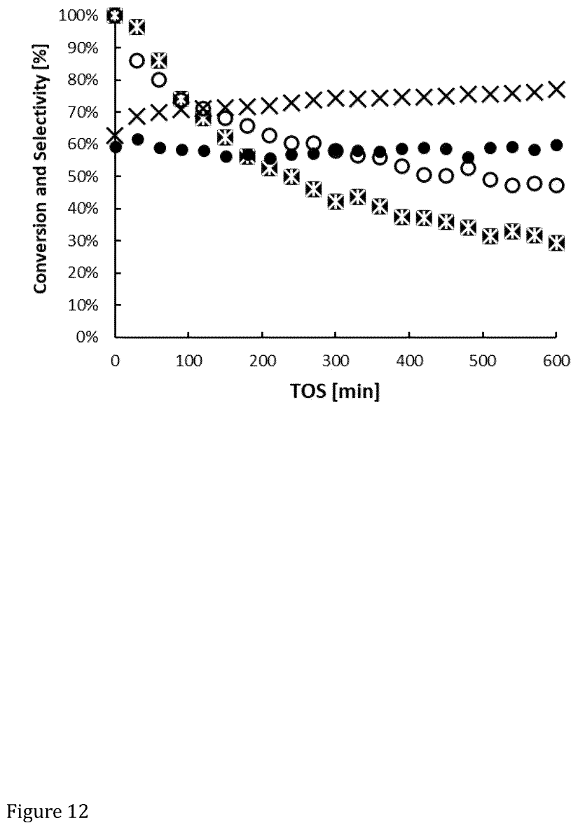

Zeolite powders excluding the impact of binders were also evaluated with ML and EL feeds. Zeolyst Na-FAU had comparable alkyl lactate conversion in ML and EL feeds, however, a maximum dehydration selectivity of only 45% was obtained over 600-min TOS with EL feed compared to 55 to 60% selectivity with ML feed (FIG. 11A). Potassium ion exchanged FAU (FIG. 11B) also exhibited similar alkyl lactate conversion between ML and EL feed type. However, dehydration selectivity was more comparable between ML and EL feed types, with a maximum of 75% for ML and a maximum of 68% for EL (FIG. 11B, triangles). Alkyl lactate conversion was enhanced in EL feed versus ML feed for K-LTL (Example 1) (FIG. 12, circles). Without being bound by theory, potassium also shows benefits for ethyl lactate feed, not just methyl lactate feed to the dehydration reactor. However, dehydration selectivity was reduced in EL feed versus ML feed for K-LTL, from a maximum of 70% to 55% (FIG. 12, triangles). Catalyst formulations of the application such as Formulation 35 or Formulation 75B have higher selectivity to dehydration than previously known materials.

Ethanol dehydration is undesirable, both from the perspective of product purification as well as related to competition with lactic species on catalyst active sites. The present formulation aims to tune catalyst structural properties such that ethanol dehydration is minimized while lactic dehydration is maximized.

### Comparative Example 17

Catalysts comprising Na-FAU powder impregnated with either 44TMDP and 12BPE amine (Example 4) showed similar feed effects as extruded catalysts and base zeolite powders. Dehydration selectivity was slightly decreased in EL feed compared to ML feed, from maximums of 78% in ML to 68% in EL for both 44TMDP and 12BPE treated Na-FAU Conversion was comparable between ML and EL feeds for 44TMDP treated Na-FAU but was decreased by approximately 20% for the 12BPE treated Na-FAU.

### Example 18

The catalytic conversion of 3-hydroxypropanoic acid (3HP) over formulation RD-0629 was evaluated using an alkyl derivative of 3HP ethyl 3-hydroxy propanoate (E3HP). E3HP feed solutions were prepared at 5 weight-% and 80 weight-% in water. 5 weight-% E3HP was fed over 24 mg of catalyst formulation 35 at 2 uL/min in 272 mL/min of N2 diluent gas. 80 weight-% E3HP was fed over 47 mg of catalyst formulation 35 at 0.5 uL/min in 272 mL/min of N2 diluent gas. Over 99% feed conversion was obtained for 6 hours on stream for 80 weight-% E3HP and for 24 hours with 5 weight-% E3HP. Over 80% dehydration selectivity was obtained for 5 weight-% 3HP and over 90% dehydration selectivity was obtained for 80 weight-% E3HP.

### Example 19

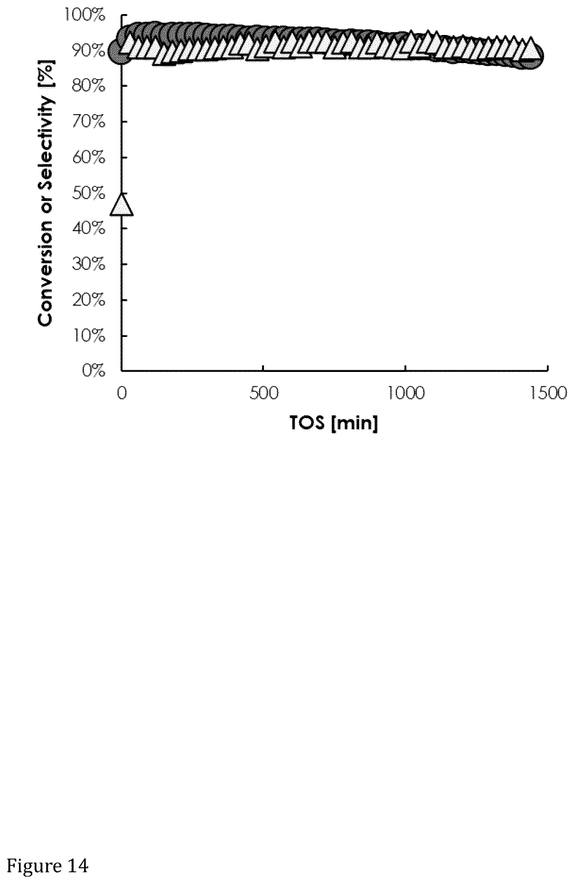

5 weight-% methyl lactate in water was fed over 24 mg of catalyst formulation 35 at 2 uL/min in 272 mL/min of N2 diluent gas. Using this feedstock and an optimized reactor geometry and feed flow regime, a 93% selectivity and an initial conversion of 94% were achieved. This is an initial yield of 87.4% which is calculated by multiplying selectivity and conversion. Even after 24 hours onstream we obtained an overall product yield of 84% as shown in FIG. 14.

### Example 20

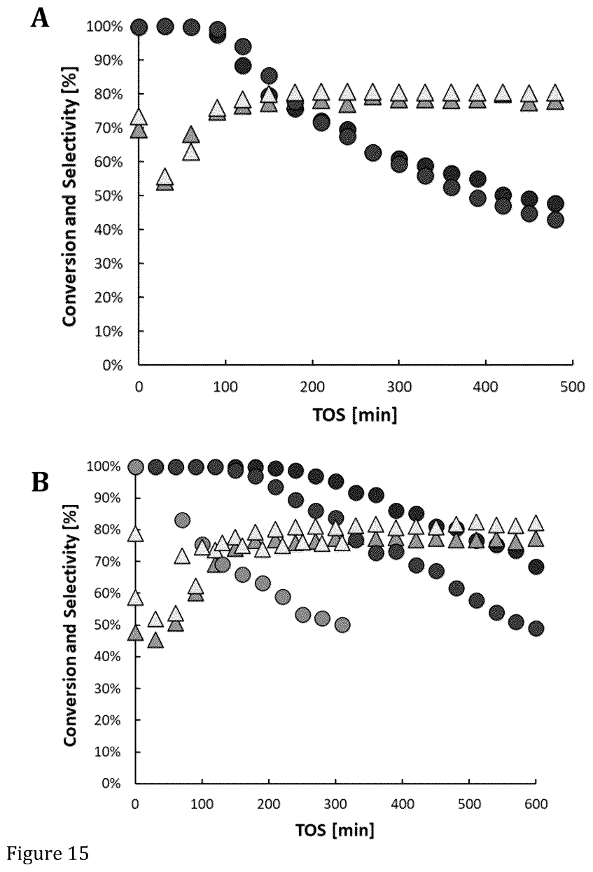

Formulations altering the mass ratios of the Na-FAU zeolite to the binder components or the relative ratios of binder components (Table 2) were prepared and tested. Formulation 21 had about 50% FAU zeolite content and Formulation 20 about 75%. Methyl lactate conversion and dehydration selectivity was near equivalent between Formulation 20 and Formulation 21, showing that over a moderate range of varying zeolite to binder ratios, catalytic performance was unchanged (FIG. 15A).

In another series, dehydration selectivity was not impacted appreciably by varying zeolite content, with selectivity of 80% achieved for formulations with high (formulation 35), moderate (Formulation 56), and low zeolite content (Formulation 59) as shown in FIG. 15B. However, conversion retention followed zeolite content, with catalyst lifetime dependent on higher zeolite concentrations in the formulation.

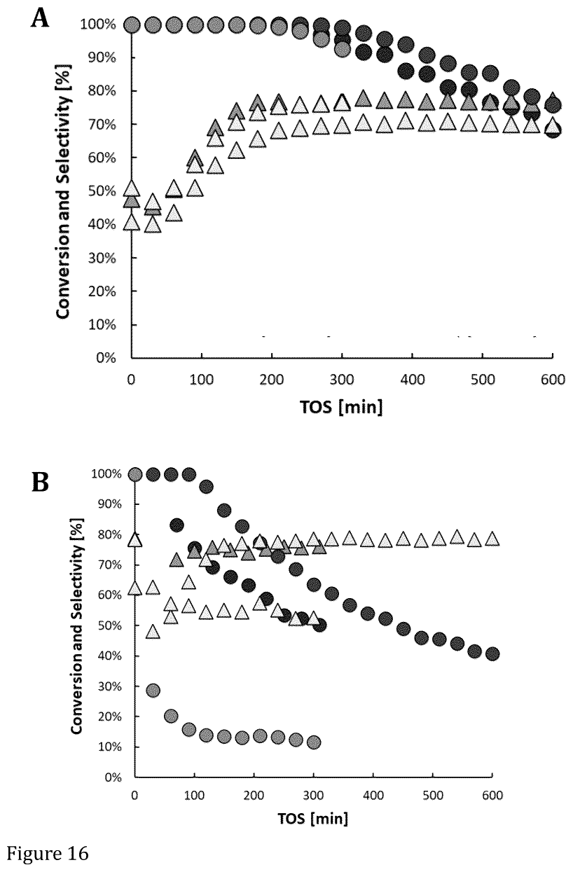

Second, the ratio of binder components in the formulation was varied while keeping zeolite content constant in the bulk formulation. This has the impact of significantly altering K/Al and total cation to Al ratios while modestly impacting Si/Al ratio. For catalysts comprising 75% zeolite, increasing K/Al from 0.28 (Formulation 60) to 0.41 (Formulation 35) resulted in no impact to methyl lactate conversion or dehydration selectivity (FIG. 16A). However, Formulation 61 with a K/Al of 0.09 exhibited a small enhancement in conversion lifetime and a small decrease in selectivity (FIG. 16A, light markers). Optimal K/Al ratios seem apparent.

As catalyst zeolite content decreases, K/Al becomes more important. For low zeolite content formulations, catalytic performance trended: Formulation 63 with a K/Al of 0.94 performed better than Formulation 59 with K/Al of 2.44 which was better than Formulation 62 with K/Al of 4.03 (FIG. 16B). Formulation 62 showed selectivity comparable to plain Na-FAU and poor conversion near 10%, while formulations 59 and 63 had selectivity comparable to other formulations with higher zeolite content. However, methyl lactate conversion for these formulations were worse than either plain Na-FAU or formulations with higher zeolite content.

### Specific Embodiments

While this invention has been described with reference to examples thereof, it shall be understood that such description is by way of illustration only and should not be construed as limiting the scope of the claimed examples. Furthermore, it is understood that the features of any example discussed herein may be combined with one or more features of any one or more examples otherwise discussed or contemplated herein unless otherwise stated.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A catalyst formulation for dehydration of lactic sources comprising:
a zeolite and at least one of one or more metal oxide sources, one or more potassium ion sources, and one or more sodium ion sources combined to form a solid acid catalyst comprising surfaces defining pores and a multiplicity of acid sites on the surfaces, wherein a total cation to aluminum ratio is greater than 1.0 and less than 1.5.

2. The catalyst formulation of claim 1 absent an amine.

3. The catalyst formulation of claim 1 wherein the zeolite comprises pore openings delimited by 12-membered rings.

4. The catalyst formulation of claim 3 wherein the zeolite comprises a FAU zeolite, an LTL zeolite, or a mixture thereof.

5. The catalyst formulation of claim 1 wherein a K/Al ratio is greater than about 0.01 and less than 1.0.

6. The catalyst formulation of claim 5 wherein an XRD peak ratio for the catalyst is greater than 1.00 and less than about 1.65.

7. The catalyst formulation of claim 5 wherein an XRD peak ratio for the catalyst are greater than 1.10.

8. The catalyst formulation of claim 1 wherein a Na/Al ratio is greater than 0.5 and less than 1.65.

9. The catalyst formulation of claim 1 wherein a Si/Al ratio is greater than 2.5 and less than 7.0.

10. The catalyst formulation of claim 1 wherein the zeolite is about 25% or more of the catalyst formulation as determined by XRD Crystallinity.

11. The catalyst formulation of claim 1 wherein the catalyst formulation is in an extruded form.

12. A process of producing an acrylic product, the process comprising:
contacting a lactic source with the catalyst formulation of claim 1 and recovering the acrylic product.

13. A method for production of a bio-based acrylic acid, the method comprising the step of:
dehydrating a reactant to yield a product by contacting a feedstream with a catalyst comprising a zeolite and at least one of one or more metal oxide sources, one or more potassium ion sources, and one or more sodium ion sources combined to form a solid acid catalyst comprising surfaces defining pores and a multiplicity of acid sites on the surfaces, wherein a total cation to aluminum ratio is greater than 1.0 and less than 1.5; the feedstream comprising a lactic source and water.

14. The method of claim 13 wherein the feedstream comprises less than about 50 wt % water.

15. The method of claim 13 wherein the feedstream comprises greater than about 50 wt % water.

16. The method of claim 13 wherein the product is one or more of an acrylic acid, an alkyl acrylate, or a cation-balanced acrylate.

17. The method of claim 13 further comprising the step of:
obtaining the lactic source from fermentation of sugar sources.

18. The method of claim 13 wherein the fermentation of sugar sources produces a mixture of two isomers or a pure lactic acid.

19. The method of claim 13 further comprising the step of:
contacting the catalyst with a second feedstream, the second feedstream comprising an alcohol.

20. The method of claim 19, wherein the second feedstream is combined with the first feedstream into a single feedstream for contacting with the catalyst.

21. The method of claim 13, wherein the contacting the feedstream with the catalyst occurs at a temperature from about 200° C. to about 350° C. by a conversion of the lactic source.

22. The method of claim 21, wherein the conversion of the lactic source may be greater than about 90 mol %.

23. The method of claim 13, wherein a yield of said acrylic acid is greater than about 84%.
